(19) United States
(12) Patent Application Publication
(10) Pub. No.: US 2012/0012104 A1
(43) Pub. Date: Jan. 19, 2012

(54) Post lipoplasty imbilical support

(76) Inventors: Yacobi Yacov, Kiriat-Ono (IL); Marcovici Iacob, Farmington, CT (US)

(21) Appl. No.: 12/804,153

(22) Filed: Jul. 15, 2010

(51) Int. Cl. A61F 5/37

(52) U.S. Cl. 128/96.1

(57) ABSTRACT

Using liposuction in abdominal cosmetic surgery gives in most cases, excellent aesthetic results. One problem associated with abdominal liposuction however, is the poor skin retraction just above the umbilicus (belly button) despite the fact that after all liposuction surgeries patients are wearing the compressive garment that is suppose to help with the skin retraction and the final satisfactory cosmetic result. However, in most cases, the umbilicus becomes covered by the surrounding abdominal skin thus, giving the umbilicus an uneven, unpleasant appearance and leaving patients and doctors alike disappointed by the final result.

To overcome this problem we propose to use of a new device called Post Lipoplasty Umbilical Support. Because of its unique shape, by applying it over the umbilical area under the traditional compressive garment immediately after surgery and during the convalescence the device will create equal radial pressure over the skin in peri-umbilical area, facilitating uniform skin retraction, round umbilicus and ultimately better cosmetic surgery results.

### BACKGROUND OF THE INVENTION

a. Field of the invention

The invention relates to a device to be use over the umbilical area (belly button) immediately after liposuction surgery and as long as the post surgery compressive garment is worn. The device is intended to better transmit the pressure generated by the post surgical garment over the problematic pen-umbilical skin area in order to improve the final cosmetic appearance of the umbilicus itself and the immediately surrounding area after liposuction surgery.

b. Description of the related art

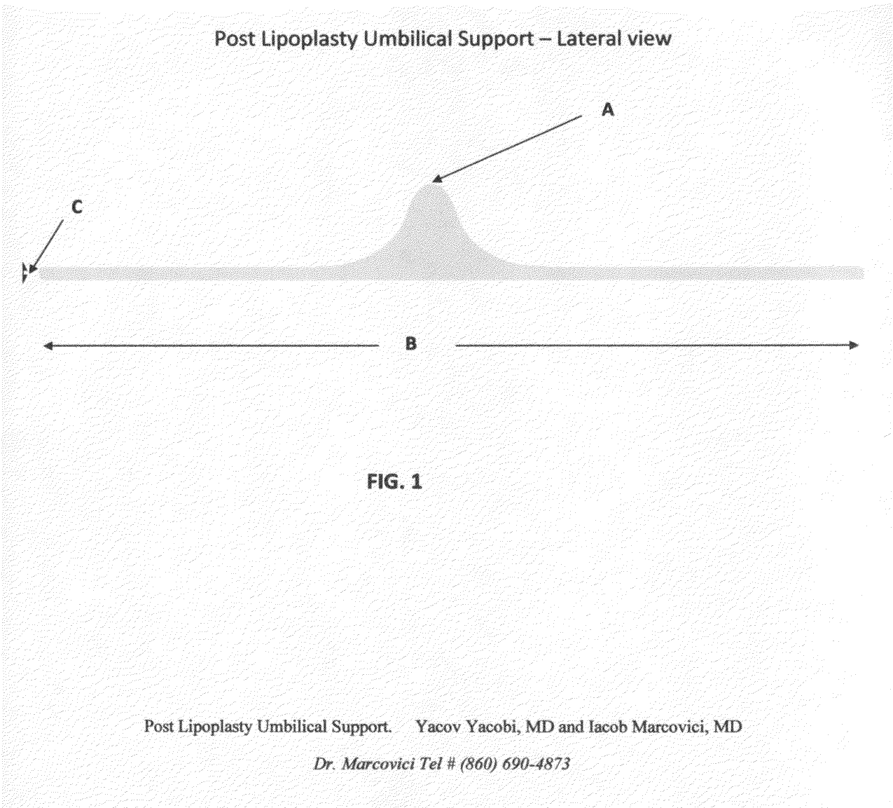

FIGS. 1 to 5 disclose a device of this kind witch comprises a rigid or semi-rigid flexible rubber disk or any material that can be applied over the umbilicus immediately after abdominal liposuction surgery. The device delivers additional skin pressure by supporting the peri-umbilical area in order to help with a better umbilical and peri-umbilical skin healing and retraction facilitating therefore, better cosmetic results.

### BRIEF SUMMARY OF THE INVENTION

The purpose of the invention is to provide a device for better cosmetic healing after liposuction surgery of the abdomen and in particular the area around the umbilicus (called also peri-umbilical area or the Belly Button area). This area presents an aesthetic challenge due to persistence of loose skin around a groove (umbilicus) caused by poor and uncontrolled retraction of peri-umbilical area after liposuction surgery. The device allows for easy treatment with great safety and within hygienic condition so important in post operative recovery period. The device helps to distribute the pressure delivered by the garment on umbilical and peri-umbilical areas in a controlled mode thus helping the surgeon and the patient to achieving the ultimate goal—a desirable cosmetic result.

### DETAILED DESCRIPTION OF THE INVENTION

The device has a central portion (A) with a height of ¼ inch and a diameter of 1/4 inch that is placed over the umbilicus and an exterior area (B) with a variable diameter (from 2 inches to 7 inches) to fit most of patients' size. The thickness of the device (C) can be a uniform ¼ inch or the upper half area (D area) can be thicker for additional pressure (and better retraction) over the upper skin around the umbilicus. The device can be made of a rigid or semi-rigid plastic or rubber of medical quality or any other comparable material.

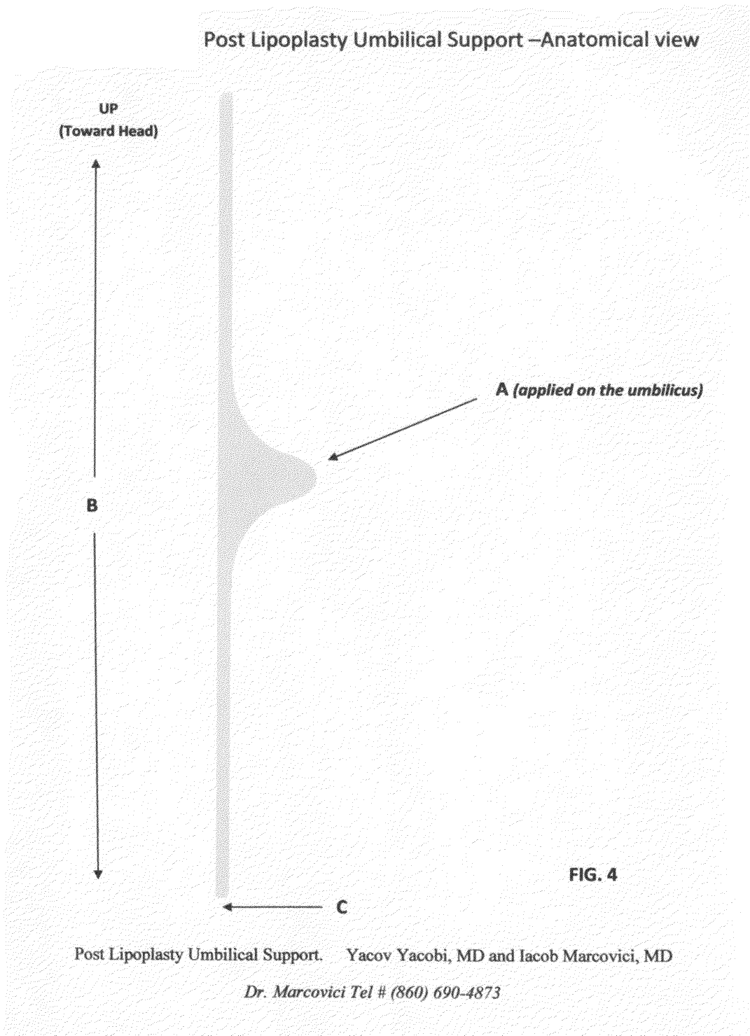

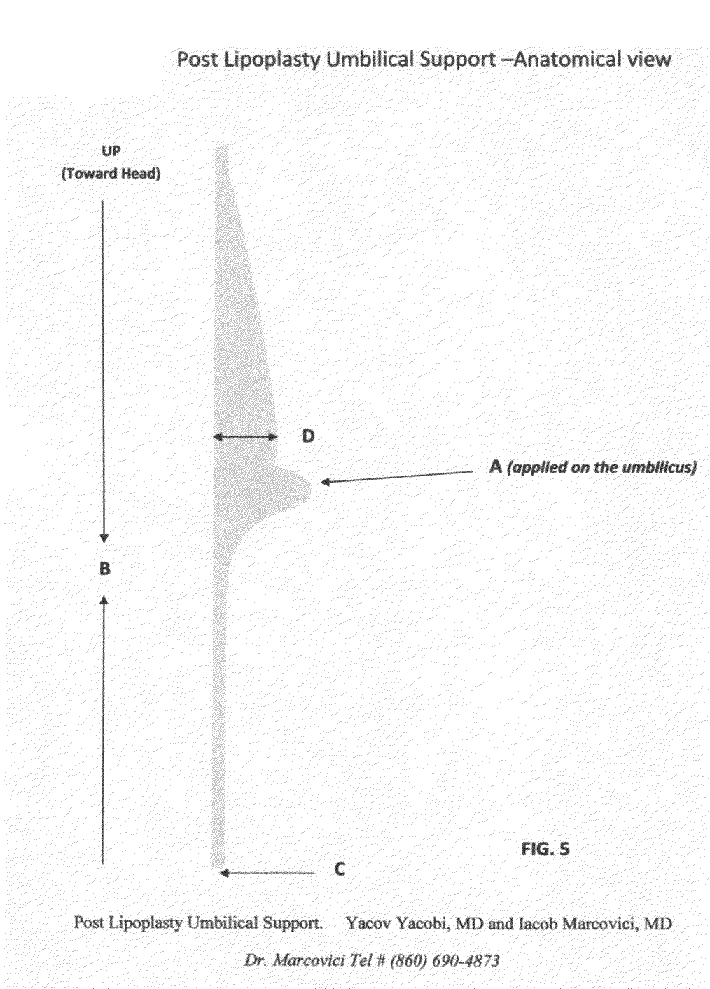

Immediately after surgery, the device is applied with the center A over the patient's umbilicus and the area B around the umbilicus as showed in FIG. 4. If the patient has a skin with poor elasticity then, the device D with its ticker upper half (as in FIG. 5) will be applied. The device is secured in place with no allergenic tape and further, is secured by the traditional compression garment that every patient is getting at the end of abdominal liposuction surgery. On some occasions, only the central portion (A) can be used-alone, applied over the umbilicus, without the B portion, for limited support to the umbilicus.

### BRIEF DESCRIPTIONS OF THE SEVERAL VIEWS OF THE DRAWINGS

### In order to describe the invention in more detail an illustrative embodiment of the device will be described reference being made to the accompanying drawings in which

FIG. 1—is a horizontal view of the device (lateral view)

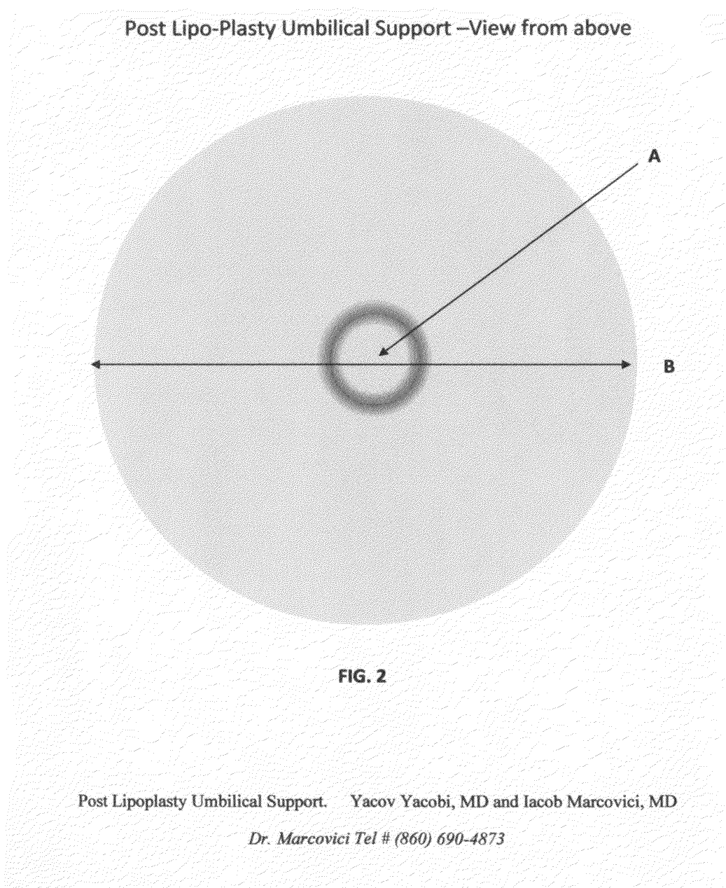

FIG. 2—is a view from above

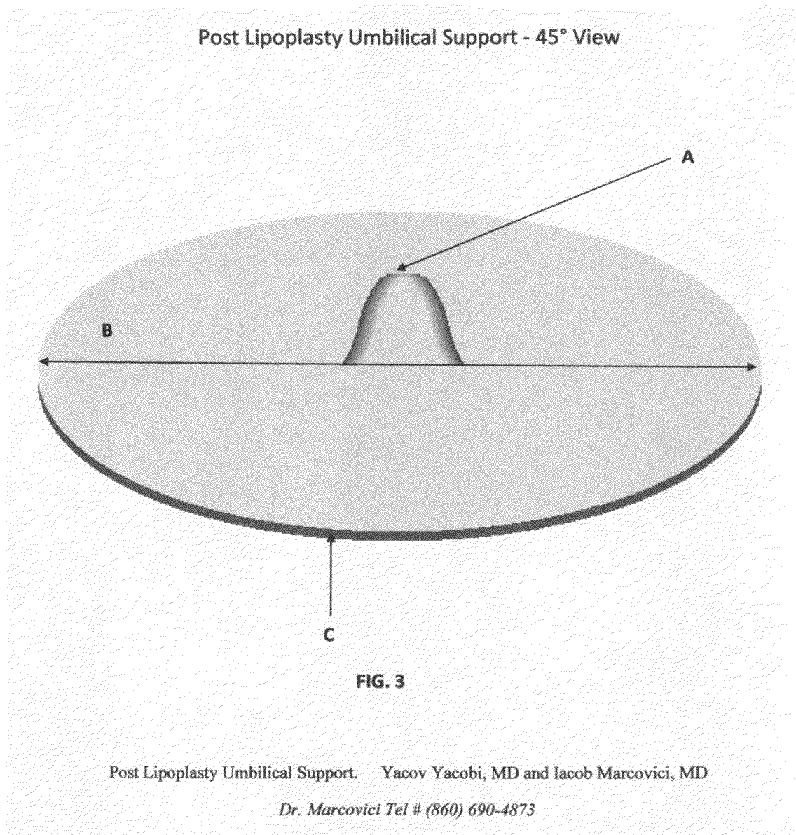

FIG. 3—is a 45° view of the device

FIG. 4—is a realistic view of the applied device

FIG. 5—is a realistic view of the applied device for patient with poor skin elasticity and in need of more skin support.

1. What we claim as our invention is that ours original proposed device, POST LIPOPLASTY UMBILICAL SUPPORT, when used as shown in FIG. 4, and/or in FIG. 5, immediately after liposuction surgery, under the post surgical garment, will solve the loose skin problem over the umbilicus and will facilitate a great cosmetic result to satisfy both patients and physicians.
